(19) United States
(12) Patent Application Publication
(10) Pub. No.: US 2007/0061165 A1
(43) Pub. Date: Mar. 15, 2007

(54) DISPLAYING PATIENT TREATMENT INFORMATION SINCE A PREVIOUS VISIT

(75) Inventors: MICHAEL A. ASH, OVERLAND PARK, KS (US); JOHN Q. DEVERTER, OVERLAND PARK, KS (US); LYNNE BEEBE, OVERLAND PARK, KS (US); HAL W. SCHIERTS, OVERLAND PARK, KS (US)

Correspondence Address: SHOOK, HARDY & BACON L.L.P.;Intellectual Property Department, 2555 GRAND BOULEVARD, KANSAS CITY, MO, 64108-2613, US

(73) Assignee: CERNER INNOVATION, INC., OVERLAND PARK, KS (US)

(21) Appl. No.: 11/426,240

(22) Filed: Jun. 23, 2006

Related U.S. Application Data
(60) Provisional application No. 60/717,294, filed on Sep. 15, 2005.

(51) Int. Cl. G06Q 10/00; A61B 5/00

(52) U.S. Cl. 705/2; 600/300

(57) ABSTRACT

Methods, computer-readable media, and user interfaces for displaying patient treatment data that is new or updated since a time a particular user last saw a particular clinical patient or last accessed patient data relevant to the patient are disclosed. Patient data in a computerized healthcare system is accessed and filtered to display only relevant information generated subsequent to a temporal reference point unique to the particular user. A user interface containing a “since last time” tab is used to display the pertinent treatment data.

### CROSS-REFERENCE TO RELATED APPLICATIONS

This application claims the benefit of U.S. Provisional Application Ser. No. 60/717,294, filed Sep. 15, 2005 and entitled “System and Method for Accessing Patient Treatment Information Generated Since a Previous Visit and Notification of Patient Treatment Information Updates,” which is hereby incorporated herein by reference. This application is related by subject matter to the invention disclosed in the commonly assigned application U.S. Application No. (not yet assigned) (Attorney Docket Number CRNI.128613), filed on even date herewith, entitled “DISPLAYING CLINICAL ORDERS AND RESULTS SINCE A PREVIOUS VISIT.”

### STATEMENT REGARDING FEDERALLY SPONSORED RESEARCH OR DEVELOPMENT

Not applicable.

### BACKGROUND OF THE INVENTION

In recent years, healthcare service providers have been making the transition from manual paper-based medical records and scheduling to an electronic format. Commercially available computer software, such as PowerChart®, PowerChart Office®, and other Cerner Millennium® solutions marketed by the Cerner Corporation of Kansas City, Mo. have advanced the state of the art well beyond the conventional manual approach. A potential problem physicians face when using this new technology is the large volume of information available to the physician for review compared to the limited amount of time the physician has to locate and analyze relevant patient information and make treatment decisions during patient visits. An electronic medical record can contain very large quantities of data pertaining to the entire medical history of the patient. Usually, the entire medical history is not needed by a physician who is about to meet with a patient. Providing too much information to the physician makes it difficult and time-consuming for the physician to sift through the record and locate the treatment data that is relevant to the patient visit.

This problem is compounded when, as is often the case, multiple physicians see a particular patient and a particular physician must determine what changes have occurred in the patient's treatment history or what new information is available since the last time that particular physician saw the patient. In an ambulatory setting, a physician needs to be able to quickly determine what has happened since the last time that physician treated the patient without being inundated by all of the information contained in the patient's electronic medical record. Providing the physician with relevant high-level information summarizing new treatment events that have occurred since the last time this physician saw the patient would allow the physician to quickly prepare for a patient visit.

A way is needed for a physician to quickly, easily, and accurately determine what patient information is new since the last time that particular physician saw the patient, without having to decipher a multitude of paper medical charts or search volumes of electronically stored data. Additionally, a physician would benefit from being able to review prior treatment decisions and diagnoses of other physicians who previously treated the patient by being able to easily view the patient's treatment information that is new since the last time that physician saw the patient.

### SUMMARY OF THE INVENTION

Embodiments of the present invention relate to methods for displaying patient treatment data that is new to a computerized healthcare system or has been updated relative to a point in time unique to a particular user. The point in time may be the time that the particular user last saw the patient for treatment purposes or last accessed the patient's data. The treatment data is displayed in sections as clinical events with associated relevant attributes.

Embodiments include filtering a patient's data to retrieve treatment data entered subsequent to a time and date stamp unique to the user. Another embodiment provides selectable regions operative to access additional information associated with selected clinical events. The additional information can be displayed as a clinical event summary that displays results relevant to the selected clinical events as well as prior results associated with prior related clinical events.

In one possible embodiment, the user interface of an electronic medical records system contains a “since last time” user interface tab that concisely displays logical sections of relevant patient treatment data generated since the prior time a particular physician saw a particular patient or accessed the patient's data. The “since last time” tab contains selectable regions operative to retrieve additional information associated with the displayed treatment data. The tab also contains page selection regions for navigating the displayed treatment data.

In another embodiment, the user interface contains a time and date stamp display area for visually providing the point in time referenced to filter the displayed treatment data. The user interface also contains a time and date stamp selection region operative to manually time and date stamp the displayed treatment data so the displayed data will not be shown to the user the next time the user accesses the “since last time” user interface tab for the particular patient.

Computer-readable media having computer executable instructions for performing embodiments of the present invention are also provided.

### BRIEF DESCRIPTION OF THE SEVERAL VIEWS OF THE DRAWINGS

The present invention is described in detail below with reference to the attached drawing figures, wherein:

FIG. 1 is a block diagram of an exemplary computing environment suitable for use in implementing the present invention;

FIG. 2 is a flow diagram showing a method for displaying patient treatment data that is new to a computerized healthcare system or has been updated since the last time a particular user saw the patient or last accessed the patient's data;

FIG. 3 is a flow diagram showing a method for displaying selected patient treatment data since the last time a particular user saw the patient or accessed the patient's data along with prior related patient treatment data;

FIG. 4 is an exemplary interactive display of a “since last time” user interface tab;

FIG. 5 is an exemplary interactive display of a “since last time” user interface tab with one clinical event selected;

FIG. 6 is an exemplary interactive display of a “since last time” user interface tab with two clinical events selected;

FIG. 7 is an exemplary interactive display of a “since last time” user interface tab with three clinical events selected;

FIG. 8 is an exemplary interactive display of a user interface containing a clinical event summary generated from lab clinical events selected by the user;

FIG. 9 is an exemplary interactive display of a “since last time” user interface tab with one document clinical event selected;

FIG. 10 is a computer screen image displaying a document report generated from a document clinical event selected by the user;

FIG. 11 is an exemplary interactive display of a user interface containing a clinical event summary pop-up window; and

FIG. 12 is an exemplary interactive display of a user interface containing a trend summary pop-up window.

### DETAILED DESCRIPTION OF THE INVENTION

The subject matter of the present invention is described with specificity herein to meet statutory requirements. However, the description itself is not intended to limit the scope of this patent. Rather, the inventors have contemplated that the claimed subject matter might also be embodied in other ways, to include different steps or combinations of steps similar to the ones described in this document, in conjunction with other present or future technologies. Moreover, although the terms “step” and/or “block” may be used herein to connote different components of methods employed, the terms should not be interpreted as implying any particular order among or between various steps herein disclosed unless and except when the order of individual steps is explicitly described.

Embodiments of the present invention provide computerized methods, computer readable media, and user interfaces for displaying a particular patient's treatment data that is new to a computerized healthcare system since the last time a particular user saw the patient. Embodiments of the present invention allow a physician or other healthcare professional to quickly identify changes that have occurred in a patient's medical history since the last time that particular physician or other healthcare professional saw the patient for treatment purposes. An exemplary operating environment is described below.

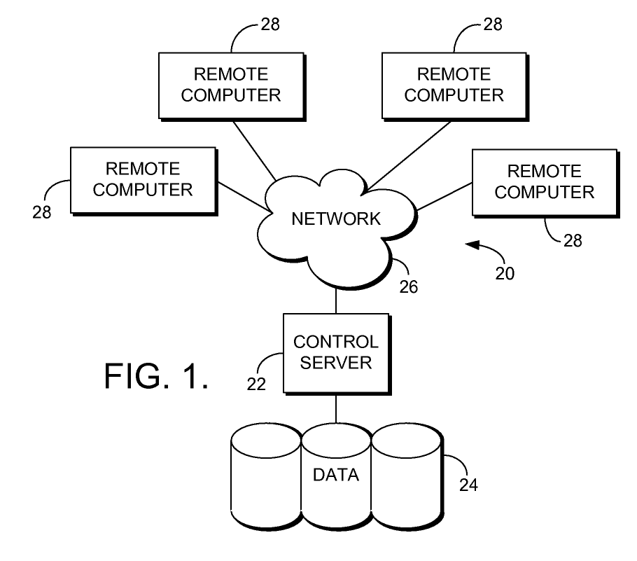

Referring to the drawings in general, and initially to FIG. 1 in particular, an exemplary computing system environment, for instance, a medical information computing system, on which the present invention may be implemented is illustrated and designated generally as reference numeral 20. It will be understood and appreciated by those of ordinary skill in the art that the illustrated medical information computing system environment 20 is merely an example of one suitable computing environment and is not intended to suggest any limitation as to the scope of use or functionality of the invention. Neither should the medical information computing system environment 20 be interpreted as having any dependency or requirement relating to any single component or combination of components illustrated therein.

The present invention may be operational with numerous other general purpose or special purpose computing system environments or configurations. Examples of well-known computing systems, environments, and/or configurations that may be suitable for use with the present invention include, by way of example only, personal computers, server computers, hand-held or laptop devices, multiprocessor systems, microprocessor-based systems, set top boxes, programmable consumer electronics, network PCs, minicomputers, mainframe computers, distributed computing environments that include any of the above-mentioned systems or devices, and the like.

The present invention may be described in the general context of computer-executable instructions, such as program modules, being executed by a computer. Generally, program modules include, but are not limited to, routines, programs, objects, components, and data structures that perform particular tasks or implement particular abstract data types. The present invention may also be practiced in distributed computing environments where tasks are performed by remote processing devices that are linked through a communications network. In a distributed computing environment, program modules may be located in local and/or remote computer storage media including, by way of example only, memory storage devices.

With continued reference to FIG. 1, the exemplary medical information computing system environment 20 includes a general purpose computing device in the form of a control server 22. Components of the control server 22 may include, without limitation, a processing unit, internal system memory, and a suitable system bus for coupling various system components, including database cluster 24, with the control server 22. The system bus may be any of several types of bus structures, including a memory bus or memory controller, a peripheral bus, and a local bus, using any of a variety of bus architectures. By way of example, and not limitation, such architectures include Industry Standard Architecture (ISA) bus, Micro Channel Architecture (MCA) bus, Enhanced ISA (EISA) bus, Video Electronic Standards Association (VESA) local bus, and Peripheral Component Interconnect (PCI) bus, also known as Mezzanine bus.

The control server 22 typically includes therein, or has access to, a variety of computer readable media, for instance, database cluster 24. Computer readable media can be any available media that may be accessed by control server 22, and includes volatile and nonvolatile media, as well as removable and nonremovable media. By way of example, and not limitation, computer readable media may include computer storage media and communication media. Computer storage media may include, without limitation, volatile and nonvolatile media, as well as removable and nonremovable media implemented in any method or technology for storage of information, such as computer readable instructions, data structures, program modules, or other data. In this regard, computer storage media may include, but is not limited to, RAM, ROM, EEPROM, flash memory or other memory technology, CD-ROM, digital versatile disks (DVDs) or other optical disk storage, magnetic cassettes, magnetic tape, magnetic disk storage or other magnetic storage device, or any other medium which can be used to store the desired information and which may be accessed by control server 22. Communication media typically embodies computer readable instructions, data structures, program modules, or other data in a modulated data signal, such as a carrier wave or other transport mechanism, and may include any information delivery media. As used herein, the term “modulated data signal” refers to a signal that has one or more of its characteristics set or changed in such a manner as to encode information in the signal. By way of example, and not limitation, communication media includes wired media such as a wired network or direct-wired connection, and wireless media such as acoustic, RF, infrared, and other wireless media. Combinations of any of the above also may be included within the scope of computer readable media.

The computer storage media discussed above and illustrated in FIG. 1, including database cluster 24, provide storage of computer readable instructions, data structures, program modules, and other data for control server 22. For example, the database cluster 24 can contain current user-specific time and date stamps assigned to portions of a patient's treatment data to designate the time and date when the particular user last accessed the portions of data. These time and date stamps can be accessed by a remote computer 28 when a particular user requests to access the portions of a patient's treatment data that are new since the last time the user accessed the data or, in other words, the portions that were generated subsequent to the time and date stamp. The database cluster 24 can also contain client-defined instructions regarding what types of patient treatment data are to be displayed to a particular user when then user requests to access the portions of data generated subsequent to the time and date stamp. The database cluster 24 can also contain an archive of prior user-specific time and date stamps assigned to portions of a patient's treatment data to designate the data displayed to the particular user at the given time and date. The stored prior stamps can be reviewed for audit purposes to determine what a particular user, such as a physician or healthcare professional, saw at a given time when the user accessed the new treatment data. This archive can be used by auditors, administrators, insurers, and others to review the patient treatment process.

The control server 22 may operate in a computer network 26 using logical connections to one or more remote computers 28. Remote computers 28 may be located at a variety of locations in a medical environment, for example, but not limited to, clinical laboratories, hospitals and other inpatient settings, ambulatory settings, medical billing and financial offices, hospital administration settings, home health care environments, and clinicians' offices. Clinicians may include, but are not limited to, a treating physician or physicians, specialists such as surgeons, radiologists and cardiologists, emergency medical technicians, physicians' assistants, nurse practitioners, nurses, nurses' aides, pharmacists, dieticians, microbiologists, and the like. Remote computers 28 may also be physically located in non-traditional medical care environments so that the entire health care community may be capable of integration on the network. Remote computers 28 may be personal computers, servers, routers, network PCs, peer devices, other common network nodes, or the like, and may include some or all of the elements described above in relation to the control server 22.

Exemplary computer networks 26 may include, without limitation, local area networks (LANs) and/or wide area networks (WANs). Such networking environments are commonplace in offices, enterprise-wide computer networks, intranets, and the Internet. When utilized in a WAN networking environment, the control server 22 may include a modem or other means for establishing communications over the WAN, such as the Internet. In a networked environment, program modules or portions thereof may be stored in the control server 22, in the database cluster 24, or on any of the remote computers 28. For example, and not by way of limitation, various application programs may reside on the memory associated with any one or all of the remote computers 28. It will be appreciated by those of ordinary skill in the art that the network connections shown are exemplary and other means of establishing a communications link between the computers (e.g., control server 22 and remote computers 28) may be utilized.

In operation, a user may enter commands and information into the control server 22 or convey the commands and information to the control server 22 via one or more of the remote computers 28 through input devices, such as a keyboard, a pointing device (commonly referred to as a mouse), a trackball, or a touch pad. Other input devices may include, without limitation, microphones, satellite dishes, scanners, or the like. The control server 22 and/or remote computers 28 may include other peripheral output devices, such as speakers and a printer.

Although many other internal components of the control server 22 and the remote computers 28 are not shown, those of ordinary skill in the art will appreciate that such components and their interconnection are well known. Accordingly, additional details concerning the internal construction of the control server 22 and the remote computers 28 are not further disclosed herein.

Although methods and systems of embodiments of the present invention are described as being implemented in a WINDOWS operating system, operating in conjunction with an Internet-based system, one of ordinary skill in the art will recognize that the described methods and systems can be implemented in any system supporting the receipt and processing of healthcare orders. As contemplated by the language above, the methods and systems of embodiments of the present invention may also be implemented on a stand-alone desktop, personal computer, or any other computing device used in a healthcare environment or any of a number of other locations.

As mentioned above, embodiments of the present invention provide computerized methods, computer readable media, and user interfaces for displaying patient treatment data that is new to a computerized healthcare system or has been updated since the last time that a particular user accessed the patient treatment data or saw the patient for treatment purposes. For simplicity, the particular user will often be referred to as the physician, but the particular user can be any healthcare professional and is not limited to a physician. Embodiments of the present invention allow a physician to quickly access the portions of a patient's treatment data that are new to the system or have been updated since the last time that particular physician saw the patient.

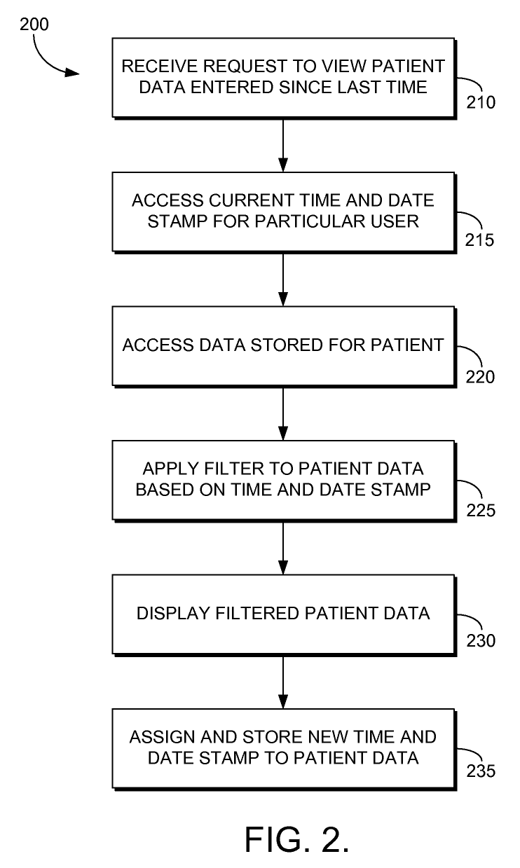

With reference to FIG. 2, an exemplary method 200 for displaying patient treatment data that is new to a computerized healthcare system or has been updated since the last time a patient was seen by a particular physician is shown. This treatment data can be obtained from a database that contains all of the patient data relevant to the particular patient. The patient data can take the form of, for example, a patient's electronic medical record (EMR). The particular user can be a physician or other health care professional who is accessing the patient's EMR for treatment purposes. For example, a physician may be preparing to meet with a patient and need to quickly determine what has happened to the patient since the last time he or she saw the patient. A method 200 can be used to obtain this desired treatment data, which provides a high-level summary of the total patient data stored in the database.

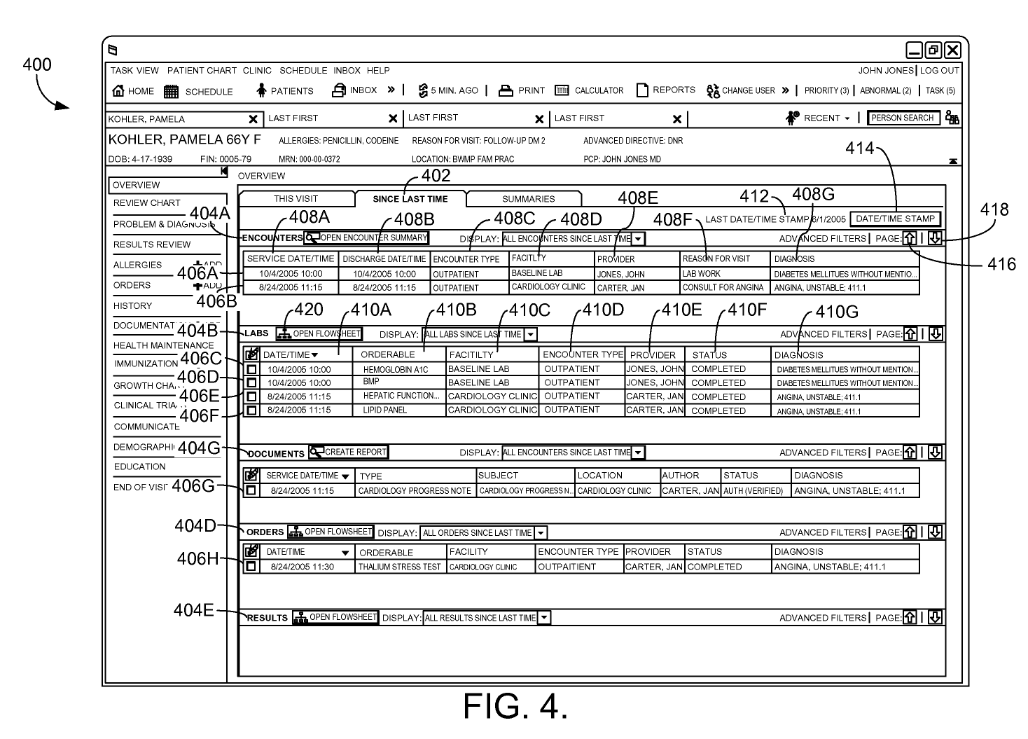

At a step 210, a request is received to view a patient's treatment data entered since the last time that the particular user saw the patient for treatment purposes or last accessed the patient's data. For example, the request may be received by the user selecting a “since last time” user interface tab. The user interface may be that of any electronic medical record system. An exemplary user interface containing a since last time tab is shown in FIG. 4 and is discussed below.

At a step 215, a time and date stamp is accessed for the particular user. The time and date stamp serves as a temporal reference point that designates the last time that the particular user saw the patient for treatment purposes or accessed the patient data. The current time and date stamp may be stored locally on a client, such as a remote computer 28 or remotely on a server, such as a control server 22. The time and date stamp is used to designate the portion of the patient data or electronic medical record that has already been previously displayed to the particular user. The portion of the patient data having an associated time and date stamp subsequent to the particular user's time and date stamp is deemed to be “new” information that must be displayed to satisfy the user's request to view the “since last time” treatment data.

For example, if Dr. John Jones last saw a patient, Pamela Kohler, on Jun. 1, 2005 at 2:00 p.m., then a time and date stamp of 2:00 p.m. on Jun. 1, 2005 had been assigned to Ms. Kohler's record for Dr. Jones. In this case, any patient data entered or updated for Ms. Kohler subsequent to 2:00 p.m. on Jun. 1, 2005 is deemed new to Dr. Jones even though it may not be new relative to other physicians who may have treated Ms. Kohler or accessed her patient data since 2:00 p.m. on Jun. 1, 2005.

At a step 220, the clinical patient's electronic medical record is retrieved and the patient's data is accessed. This could involve accessing locally stored data or accessing remotely stored data to obtain patient data stored for the patient in the system. The patient data are filtered to obtain the desired treatment data, because the portion of the patient data with an associated creation time or update time subsequent to the particular user's time and date stamp are the desired treatment data.

At a step 225, one or more filters are applied to the patient data to extract the treatment data, which is the information that is subsequent to the current time and date stamp for the particular user. In the alternative, a bookmark or series of bookmarks could be used as the temporal reference point to designate the new or updated treatment data of interest to the particular user. The temporal reference point is used to filter the patient data. For example, on Oct. 31, 2005, Dr. Jones is preparing for a visit with a patient, Ms. Kohler, and requests to view the portion of Ms. Kohler's patient data that is new or has been updated since the last time he saw Ms. Kohler. If Dr. Jones last saw her on Jun. 1, 2005, a time and date stamp of Jun. 1, 2005 exists for Dr. Jones, the particular user. To satisfy Dr. Jones' request, Ms. Kohler's patient data are filtered to retrieve the treatment data of interest, the data entered between Jun. 1, 2005 and Oct. 31, 2005. This is done by retrieving the data with a creation (or update) time and date subsequent to the Jun. 1, 2005 time and date stamp.

At a step 230, the filtered patient data (i.e., the treatment data) are displayed. The treatment data can be categorized and displayed by configurable user-defined instructions. In this embodiment, a particular categorization scheme is applied to the patient data and will be described in further detail below with reference to FIG. 4. A user interface that contains a “since last time” user interface tab can be used. In this embodiment, the treatment data is displayed in a “since last time” user interface tab according to the categorization scheme.

At a step 235, a new time and date stamp is assigned to the displayed patient treatment data and stored. The new time and date stamp designates that this portion of the patient's electronic medical record has been viewed during this visit and should not be displayed in the future under the “since last time” tab the next time that the particular user requests to view the since last time treatment data. The assignment of the new time and date stamp can be automatic or can be performed manually upon receiving a request to assign a new time and date stamp, such as by the user selecting a time and date stamp selection region. The new time and date stamp can be stored locally or remotely.

For example, after viewing Ms. Kohler's treatment data, which was new or updated since her last visit with Dr. Jones, and meeting with Ms. Kohler on Oct. 31, 2005, Dr. Jones may assign a new time and date stamp of Oct. 31, 2005, unique to him, to her record. This may be done by receiving a selection via a time and date stamp selection region or it may occur automatically upon Dr. Jones viewing the since last time treatment data. In this example, the next time Dr. Jones views Ms. Kohler's record, patient data subsequent to Oct. 31, 2005 will appear in the since last time user interface tab of her record. In this embodiment, prior time and date stamps corresponding to previous visits can be stored in an archive to allow the particular physician user, auditors, or other individuals to view what information was displayed in the since last time tab for that particular visit.

After completion of the exemplary method 200, the user can quickly determine any changes that have occurred in the patient's treatment history since the last time that he or she saw this patient or accessed the patient's data. The method 200 eliminates noise from the large quantity of data that is stored in the patient's electronic medical record. This noise, or extraneous data, is not relevant to a physician about to meet with a patient. The physician can, of course, still review the patient's entire electronic medical record, if necessary. The method 200 displays a concise view summarizing the new or updated treatment events occurring in the patient's history since the last time this physician saw the patient. Additionally, the physician may obtain more detailed information about these new or updated treatment events by interacting with the “since last time” user interface tab, as discussed below.

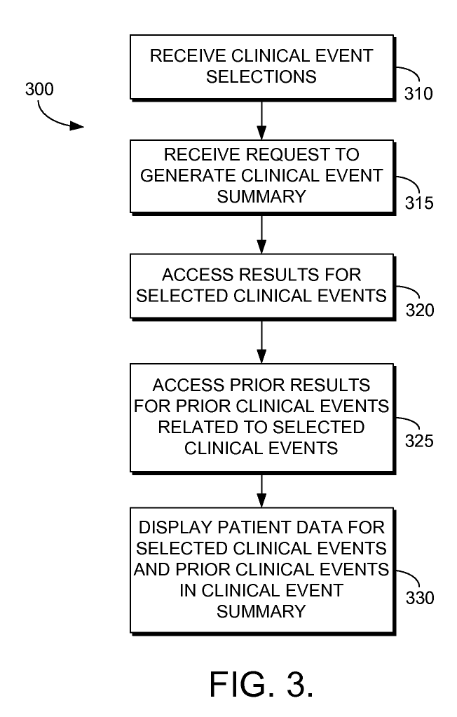

With reference to FIG. 3, an exemplary method 300 for displaying additional information relevant to selected clinical events and prior related clinical events is shown. A physician viewing high-level information regarding a patient's medical history since the last time the physician saw the patient, may wish to view more detailed information related to the high-level information. For example, Dr. Jones may notice that his patient, Ms. Kohler, had some laboratory tests done since the last time he saw her. He may wish to obtain more detailed information about the tests and their results.

At a step 310, one or more clinical event selections is received. A clinical event may be any patient treatment event, such as, for example, a patient visit encounter, a laboratory test or panel of tests, a radiology event, medications ordered or taken, ordered or performed procedures, administrative events, documents relevant to the patient's treatment, brief notes, comments, or any other item pertaining to treatment or care of the patient. A clinical event selection may be received by a user selecting an associated selectable region corresponding to the clinical event. The clinical event selection can take the form of, for example, placing a check in a box or highlighting the clinical event. In this embodiment, multiple clinical event selections may be made within a section. For example, several laboratory tests may have been conducted since the physician last saw the patient and the physician may want to access results for all of these tests. The physician might select a clinical event corresponding to a hemoglobin A1C test and a clinical event corresponding to a lipid panel test that were performed since the last time the physician saw the patient.

At a step 315, a request is received to generate a clinical event summary, such as a flowsheet or other results summary providing additional information regarding the clinical event selections. For example, the physician may want to view a summary of the results from the selected hemoglobin A1C and lipid panel tests referred to in the above example. In addition, the physician would probably also be interested in laboratory results from any prior hemoglobin A1C and lipid panel tests, as well as any other prior laboratory test results from tests typically ordered for the same reasons that hemoglobin A1C and lipid panel tests are usually ordered. For example, if a user requests to view a clinical event summary of results of a lipid panel test, which includes cholesterol and triglyceride levels, prior results from other similar tests that also contain results for cholesterol and triglyceride levels may also be relevant to the physician's request. In an embodiment, the clinical event summary can also display such prior results from prior clinical events having a related attribute (e.g., cholesterol and triglyceride levels) associating the prior clinical events with the selected clinical events.

At a step 320, the patient data corresponding to results for the selected clinical events are accessed. And, at a step 325, the patient data corresponding to prior results for the prior clinical events having a related attribute associating the prior clinical events with the selected clinical events are also accessed. For example, the results for the selected hemoglobin A1C and lipid panel tests are accessed as well as prior results for prior hemoglobin A1C, lipid panel, and related tests, such as those discussed in the above example.

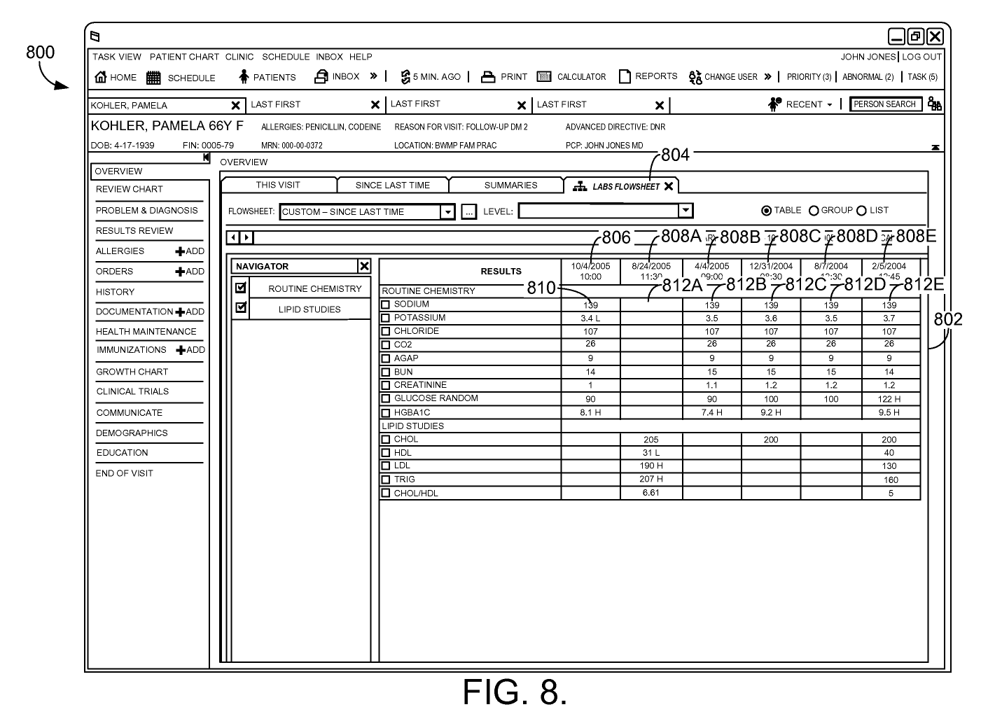

At a step 330, results from the selected clinical events and prior results from the prior clinical events are displayed in a flow sheet or other summary format. The results associated with the selected laboratory test clinical events are displayed along with the historical results associated with the prior related laboratory test clinical events. In this embodiment, the results for the selected laboratory tests are displayed along with the results from five prior laboratory tests. This will be discussed in further detail below with reference to FIG. 8. The juxtaposition of the displayed results for the selected tests with the displayed prior results allows a physician to view any trends that may exist. In some embodiments, a clinical event summary for a single selected clinical event or for multiple selected clinical events can be displayed. In other embodiments, a clinical event summary for prior related clinical events can be displayed. And, in other embodiments, a clinical event summary for both selected clinical events and prior related clinical events can be displayed.

After completion of an exemplary method 300, additional detailed information associated with clinical events located in a “since last time” user interface tab has been displayed satisfying the physician's request for lower level information pertaining to the patient.

In FIG. 4, an exemplary user interface 400 for performing embodiments of the present invention is shown. In one embodiment, exemplary user interface 400 contains a “since last time” user interface tab 402. In an embodiment of the present invention, tab 402 serves as a selection region operative to receive requests from a particular user to view a patient's treatment data that is new to a computerized healthcare system or has been updated since the patient was last seen by the particular physician user or since the particular physician user last accessed the patient's data. User interface 400 contains sections 404A-404E that categorize clinical events 406A-406H. The sections 404A-404E may be, for example, encounters, labs, documents, orders, results, and any other logical sections to categorize the treatment data including, but not limited to, sections corresponding to the other examples of types of clinical events described above.

In one embodiment, encounter clinical events 406A-406B represent patient visit encounters. Attributes 408A-408G containing information relevant to each clinical event are displayed for each clinical event. Attributes may vary depending on the section. For example, a patient visit clinical event 406A within encounter section 404A may have a date and time attribute 408A, a discharge date and time attribute 408B, and an encounter type attribute 408C, as well as other attributes. These attributes contain information relevant to the clinical event. For example, some of these attributes describe the date and time of the patient visit, the date and time the patient was discharged, and the type of patient visit, such as an outpatient encounter. The attributes displayed for a particular section may be based on a configurable pre-defined database. In this embodiment, a particular categorization scheme has been employed.

Within a labs section 404B, laboratory test clinical events 406C-406F corresponding to tests performed since the last time the physician saw the patient are displayed. In this embodiment, the attributes 410A-410G associated with laboratory test clinical events 406C-406F provide details for the particular laboratory test clinical events. For example, laboratory test clinical event 406C corresponds to a hemoglobin A1C test performed on Oct. 4, 2005 at 10:00 at the baseline lab facility, for example.

In one embodiment, each section has a selectable region 420 for receiving a request to display additional information about the clinical events within that section. Each clinical event within a section has an associated selectable region. For example, labs section 404B has an “open flowsheet” selection region 420 that is operative to generate and display a flow sheet containing information associated with any lab clinical events 406C-406F that are selected by the user. This will be discussed in further detail below.

In an embodiment, “since last time” user interface tab 402 also contains a time and date stamp display area 412 indicating the date and time stamp assigned to the particular user. Display area 412 further indicates the stamp that was used during filtering of the patient data, which yielded the treatment data displayed in tab 402. The time and date stamp was discussed above with reference to FIG. 2. In this embodiment, the “since last time” tab 402 contains a date and time stamp selection region 414 operative to receive requests from the user to manually time and date stamp the patient's electronic medical record for the date and time at which the user is viewing user interface 400. The manual time and date stamp allows a user to control when the displayed treatment data is re-assigned a new time and date stamp. This allows a user to address other matters or to switch to a new display to access other electronic records without a new automatic time and date stamp being assigned to the displayed treatment data. This manual approach is an alternative to automatic time and date stamping, which also may be used, as discussed above.

In one embodiment, each section contains page selection regions 416 and 418. In this embodiment, each section contains a page up selection region 416 and a page down selection region 418 operative to reconfigure interface 400 to display other additional clinical events within each section. For example, a user may want to view a patient visit clinical event occurring before the patient visit clinical event 406B and could do so by selecting page down selection region 418. The user interface tab 402 can be configured to display more or fewer clinical events under each section. For example, labs section 404B displays four lab clinical events 406C-F; this section can be configured to display more or less than four clinical events. The page up and page down selection regions allow the user to view additional clinical events, and, at the same time, the page up and page down selection regions facilitate a concise representation of data. A concise representation allows the physician to be quickly and efficiently updated about a patient's case prior to a patient visit without being overloaded with information.

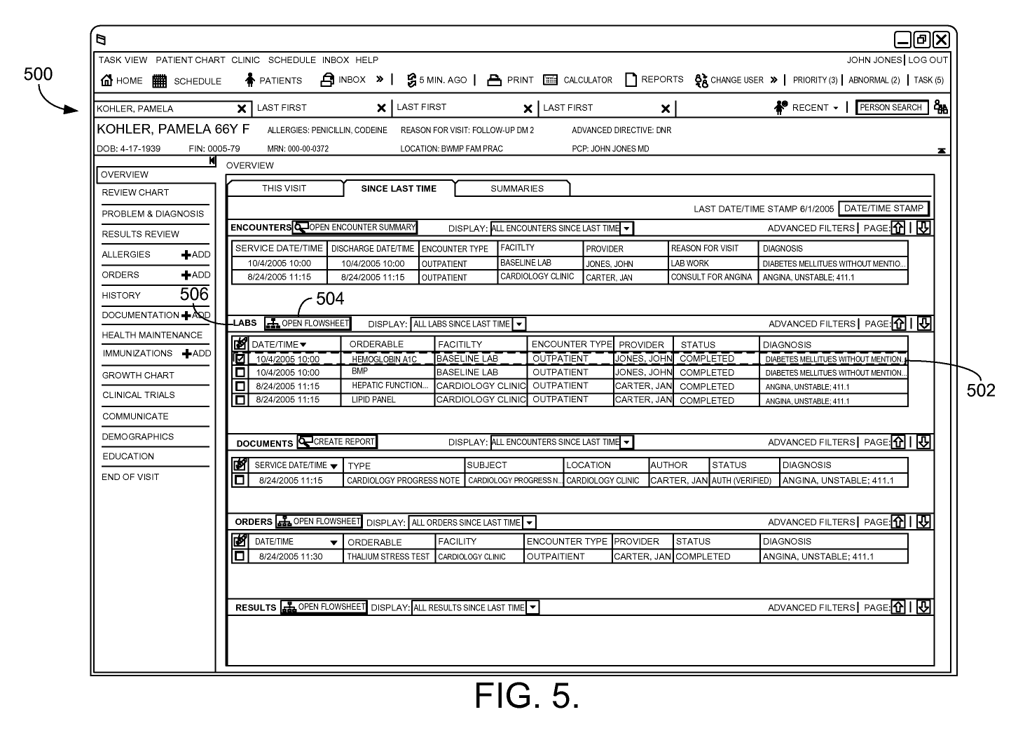

In FIG. 5, an exemplary user interface 500 for performing an embodiment of the present invention is shown. User interface 500 displays a clinical event selection region 502 corresponding to a selected clinical event. In this embodiment, for example, a hemoglobin A1C laboratory test clinical event has been selected by the particular user. Similarly, clinical events under any of the sections may be selected in a like manner. A selectable region 504 located within a section 506 may subsequently receive a request to display additional information associated with the selected clinical event. As discussed above with reference to FIG. 3, a clinical event summary can be generated for the selected clinical event. A request to generate a clinical event summary is initiated by the user selecting the “open flowsheet” selectable region 504.

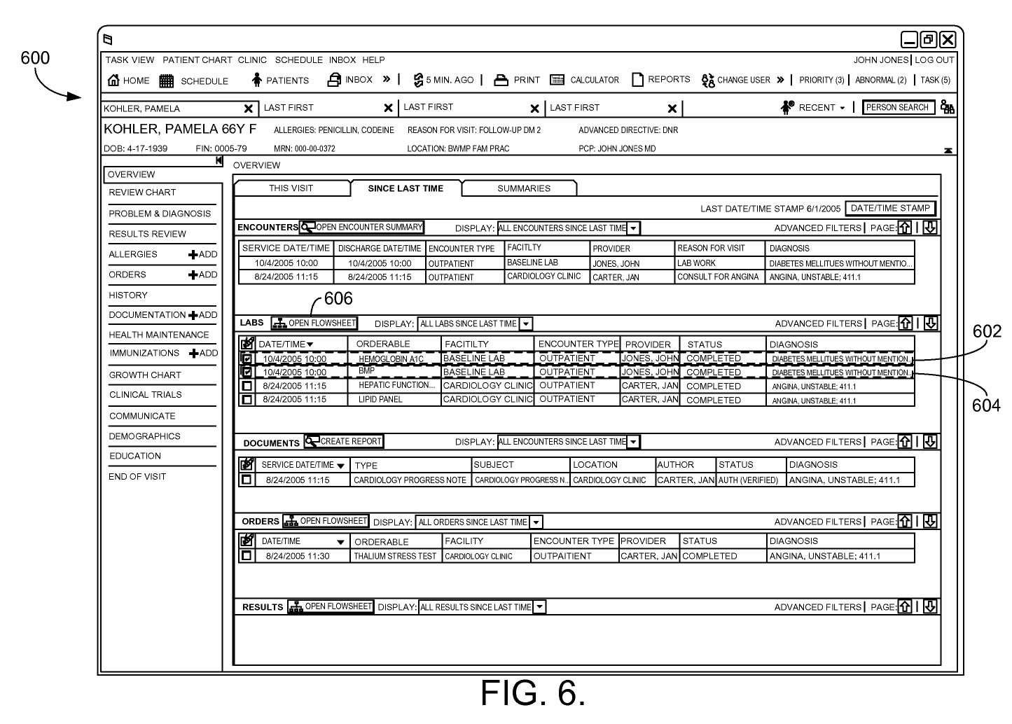

As shown in FIG. 6, an exemplary user interface 600 can display selected clinical events 602 and 604 in order to facilitate retrieval of additional information for multiple clinical events at the same time. The open flow sheet selectable region 606 can subsequently be used to receive requests to display additional information associated with selected clinical events 602 and 604.

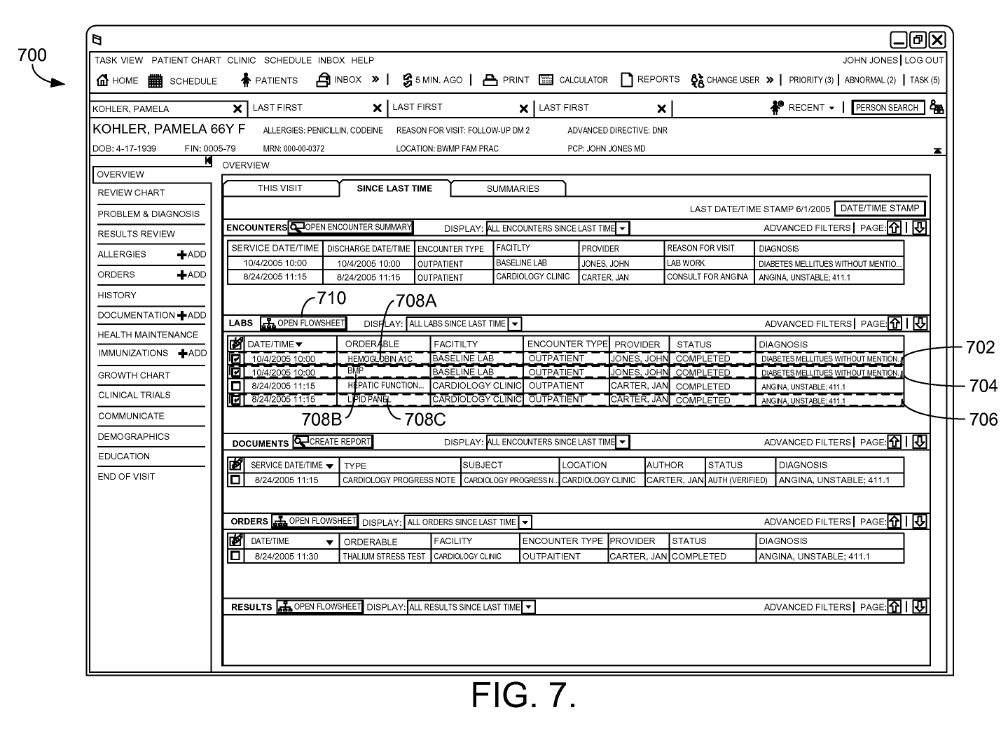

As shown in FIG. 7, an exemplary user interface 700 can display multiple selected clinical events 702, 704, and 706 corresponding to laboratory test clinical events having laboratory tests type attributes 708A, 708B, and 708C of hemoglobin A1C, BMP, and lipid panel, respectively. Upon selection of an open flow sheet selectable region 710, a flow sheet can be generated to display results associated with the selected laboratory test clinical events. This process was described above with reference to FIG. 3.

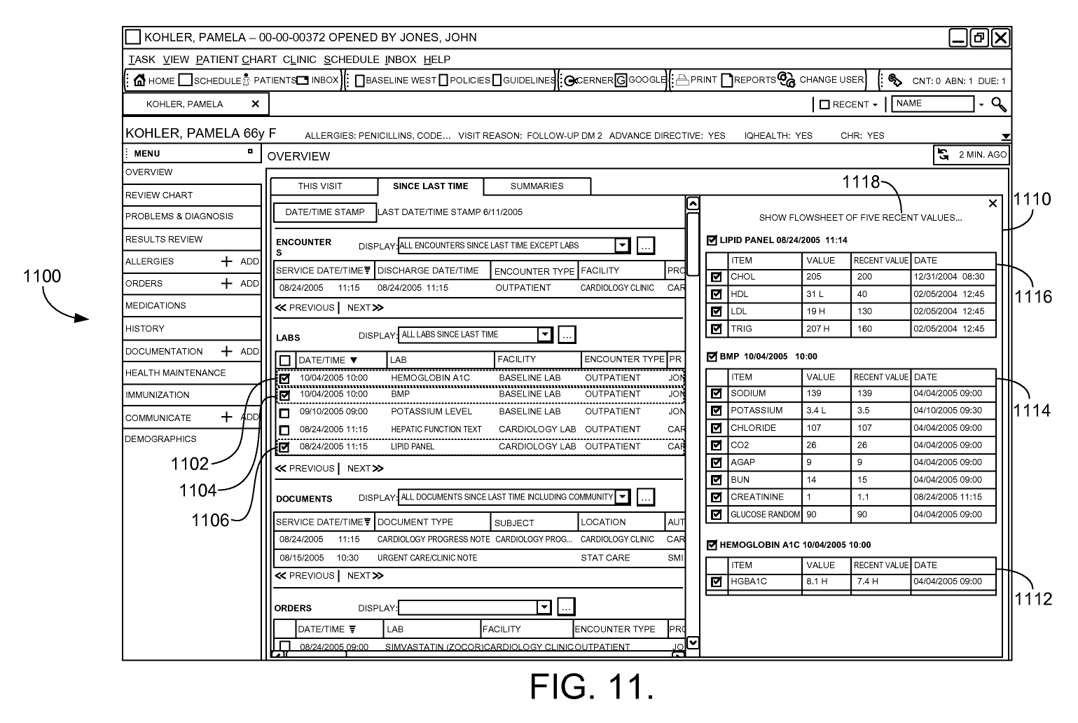

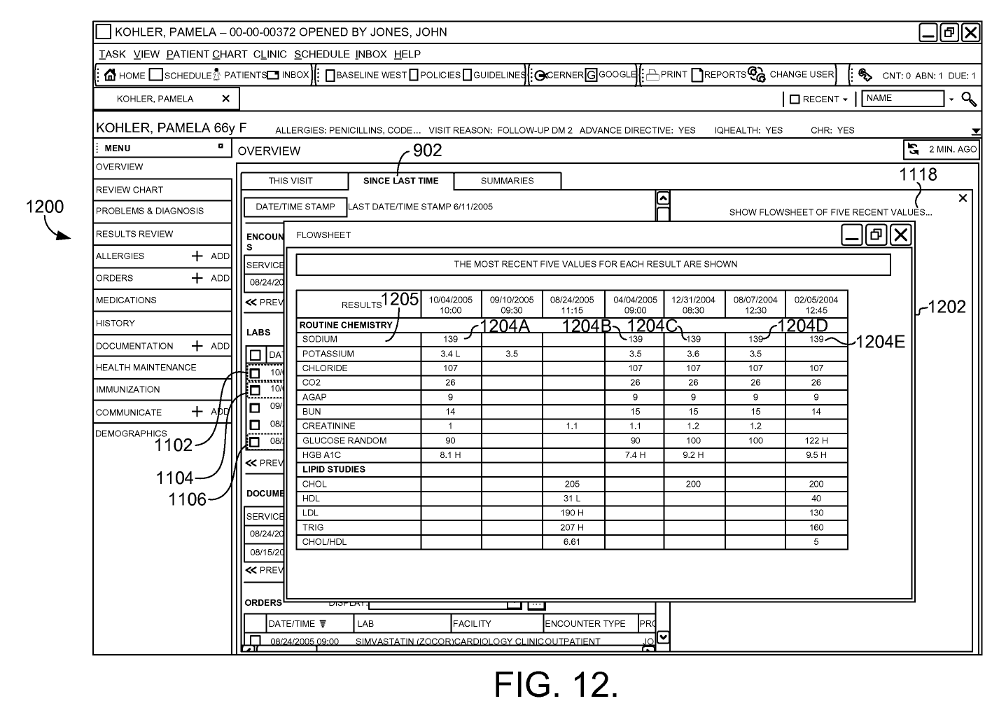

Referring to FIG. 8, an exemplary user interface 800 is shown for displaying a generated clinical event summary 802. The clinical event summary 802 is displayed within a clinical event summary tab 804. The clinical event summary 802 displays results 810 associated with the selected clinical event 806. In this case, clinical event summary 802 is a labs flowsheet containing laboratory test results for selected laboratory test clinical events. A clinical event summary can also be displayed in the form of a trend summary pop-up window that appears simultaneously on a common display with sections, clinical events, and attributes. An example of this will be provided with reference to FIGS. 11-12 below. User interface 800 also includes prior results 812A-812E associated with prior clinical events 808A-808E. In this embodiment, user interface 800 displays prior laboratory test results associated with prior laboratory test clinical events. The particular user has requested to view laboratory test results for a hemoglobin A1C and lipid panel laboratory test conducted for the patient. The hemoglobin A1C test has various results associated with it such as sodium levels, potassium levels, chloride levels, etc. Since a user requested to view a selected hemoglobin A1C laboratory test clinical event in a flow sheet, the five previous hemoglobin A1C test clinical events are also displayed with their associated results including the patient's prior levels of sodium, potassium, chloride, etc. In this embodiment, results associated with a selected lipid panel test clinical event are also displayed. As shown in user interface 800, prior results from prior lipid panel clinical events for this patient are also displayed. The user interface allows a physician to quickly review any potential trends that may exist in the result values. In this embodiment, user interface 800 can also display prior results from prior related laboratory test clinical events that are requested for the same or similar reasons as the selected laboratory tests. For example, if a user requests to view a clinical event summary of results of a lipid panel test, which includes cholesterol and triglyceride levels, prior results from other similar tests that also contain results for cholesterol and triglyceride levels can also be displayed.

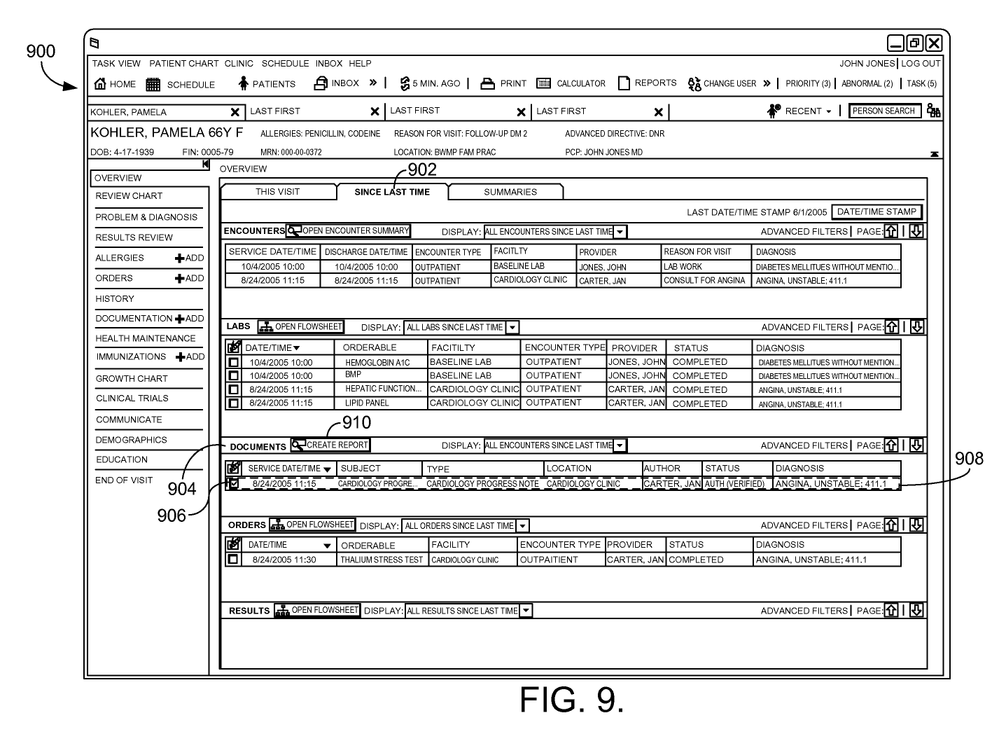

Referring to FIG. 9, an exemplary user interface 900 is shown for displaying patient treatment data generated or updated since the last time the user saw the patient or accessed the patient's data. In user interface 900, a “since last time” user interface tab 902 contains a documents section 904, which has a document clinical event 906. A document clinical event can be an brief electronic note, a medical report, a comment, a progress note, an administrative document, or any other electronic document relevant to the patient. Documents section 904 contains a selectable region 910 operative to receive a request to display additional information associated with a selected document clinical event 908. Selectable region 910 can be a “create report” selectable region.

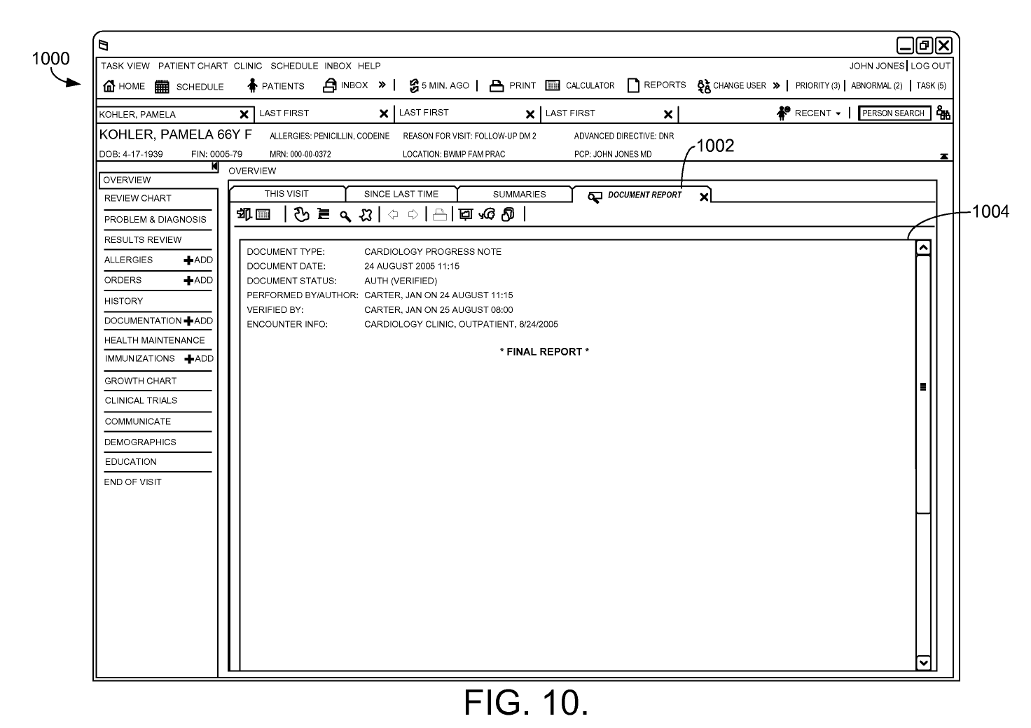

Upon receiving a request to create a report for a document clinical event selection, the system displays a user interface 1000, as shown in FIG. 10. User interface 1000 contains a document report tab 1002 that contains additional information associated with the selected document clinical event 906. Document report tab 1002 contains a results display region 1004 containing results describing the selected document clinical event. For example, the results display region 1004 can contain additional information, such as the type of document, the date the document was generated, the status of the document, the author of the document, the contents of the document, and other related additional information.

As shown in FIG. 11, an exemplary user interface 1100 displays clinical event selection regions within each section that are operative to receive requests to automatically generate a clinical event summary pop-up window summarizing selected clinical events. In this case, user interface 1100 displays selected clinical events 1102, 1104, and 1106 corresponding to a hemoglobin A1C test, a BMP, and a lipid panel test that are new to the system relative to a time and date stamp corresponding to the last time the user saw the patient for treatment purposes or accessed the patient's EMR. Upon selection of clinical events 1102, 1104, and 1106, a clinical event summary pop-up window 1110 is displayed in user interface 1100. Clinical event summary pop-up window 1110 automatically displays results associated with each selected clinical event simultaneously. In this embodiment, the results associated with each selected clinical event are shown, as well as prior results obtained for the last clinical event of the same type. For example, a current cholesterol level of 205 is displayed for a lipid panel test conducted on Pamela Kohler on Aug. 24, 2005 at 11:14. In addition, a prior cholesterol level of 200 is displayed for a lipid panel or other cholesterol-determining test conducted on Pamela Kohler on Dec. 31, 2004 at 8:30. A trend selection region 1118 can be displayed in pop-up window 1110. Trend selection region 1118 is operative to receive a request to display results from the selected clinical events 1102, 1104, and 1106 as well as prior results obtained from five previous clinical events of the same type. Upon selection of trend selection region 1118, a user interface displaying these results, such as user interface 1200, is shown.

As shown in FIG. 12, an exemplary user interface 1200 displays a trend summary pop-up window 1202. In this embodiment, pop-up window 1202 displays laboratory test results for Pamela Kohler that are relevant to selected laboratory clinical events 1102, 1104, and 1106, which are new to the system relative to a time and date stamp corresponding to the last time the patient was seen for treatment purposes by the user or the last time the user accessed the patient's data. The results from the selected clinical events, as well as up to the five most recent values 1204A-1204E for each particular result 1205 are displayed. In alternative embodiments, the number of results displayed may vary depending on the relevant number of results typically required to recognize a trend. Pop-up window 1202 is displayed simultaneously on a common display with a “since last time” tab 902 containing sections categorizing clinical events, such as lab results, orders, patient visit encounters, documents, and medications, as well as attributes associated with each clinical event.

The present invention has been described in relation to particular embodiments, which are intended in all respects to be illustrative rather than restrictive. Alternative embodiments will become apparent to those of ordinary skill in the art to which the present invention pertains without departing from its scope.

From the foregoing, it will be seen that this invention is one well adapted to attain all the ends and objects set forth above, together with other advantages which are obvious and inherent to the system and method. It will be understood that certain features and sub-combinations are of utility and may be employed without reference to other features and sub-combinations. This is contemplated by and within the scope of the claims.

The invention claimed is:

1. A method in a computerized healthcare system for displaying treatment data relevant to a clinical patient, the method comprising:
displaying a plurality of sections that categorize the treatment data, wherein the treatment data is new to the system or has been updated relative to a temporal reference point unique to a particular user and further wherein the treatment data comprises at least one clinical event;
displaying the clinical event within at least one of the sections; and
displaying, for the clinical event, relevant attributes that provide information regarding the clinical event;
wherein the sections, clinical event, and attributes are displayed simultaneously.

2. The method of claim 1, further comprising:
accessing a database containing patient data relevant to the clinical patient; and
filtering the patient data to identify the treatment data new to the system relative to the temporal reference point.

3. The method of claim 2 wherein the temporal reference point corresponds to a time that prior data relevant to the clinical patient was last accessed by the particular user.

4. The method of claim 2 wherein the temporal reference point corresponds to a time that the clinical patient was last seen for treatment purposes by the particular user.

5. The method of claim 4 wherein the temporal reference point is determined by a time and date stamp.

6. The method of claim 5, further comprising:
assigning a new time and date stamp to the displayed treatment data, wherein the new time and date stamp is unique to the particular user; and
storing the new time and date stamp.

7. The method of claim 4 wherein the plurality of sections comprises a section categorizing laboratory tests for the clinical patient and a section categorizing patient treatment encounters for the clinical patient.

8. The method of claim 4 wherein the clinical event has at least one associated selectable region such that upon selection, additional information relevant to the clinical patient is displayed.

9. The method of claim 8, further comprising:
receiving a request to generate a clinical event summary comprising at least one result relevant to at least one selected clinical event; and
displaying the clinical event summary.

10. The method of claim 9 wherein the clinical event summary further comprises at least one prior result relevant to at least one prior clinical event having a related attribute associating the prior clinical event with the selected clinical event.

11. The method of claim 9 wherein the result is at least one laboratory test result and the selected clinical event is at least one laboratory test clinical event.

12. The method of claim 10 wherein the prior result is at least one prior laboratory test result, the prior clinical event is at least one prior laboratory test, and the related attribute is a related laboratory test type.

13. The method of claim 10 wherein no more than five prior clinical events are displayed.

14. The method of claim 12 wherein no more than five prior laboratory test clinical events are displayed.

15. One or more computer readable media having computer executable instructions embodied thereon for performing a method for displaying treatment data relevant to a clinical patient, the method comprising:
accessing a database containing patient data relevant to the clinical patient;
filtering the patient data to identify the treatment data, wherein the treatment data is new to the system or has been updated relative to a temporal reference point unique to a particular user and further wherein the treatment data comprises at least one clinical event;
displaying a plurality of sections that categorize the treatment data; and
displaying, for the clinical event, relevant attributes that provide information regarding the clinical event;
wherein the sections, clinical event, and attributes are displayed simultaneously.

16. The computer readable media of claim 15, wherein the temporal reference point corresponds to a time that the clinical patient was last seen for treatment purposes by the particular user or a time that the patient data relevant to the clinical patient was last accessed by the particular user.

17. The computer readable media of claim 16, wherein the temporal reference point is determined by a time and date stamp.

18. The computer readable media of claim 17, wherein the clinical event has at least one associated selectable region such that upon selection, additional information relevant to the clinical patient is displayed.

19. A user interface embodied on at least one computer readable medium, the user interface comprising a display showing:
treatment data relevant to a clinical patient, wherein the treatment data is new or updated relative to a temporal reference point unique to a particular user and further wherein the treatment data comprises at least one clinical event;
a plurality of sections that categorize the treatment data; and
attributes relevant to the clinical event that provide information regarding the clinical event;
wherein the treatment data, sections, and attributes are shown simultaneously.

20. The user interface of claim 19, further showing at least one clinical event selection region operative to receive a clinical event selection.

21. The user interface of claim 20, further showing, for each section, at least one page selection region operative to receive a request to display at least one additional clinical event associated with a section.

22. The user interface of claim 21, wherein the temporal reference point corresponds to a time that the clinical patient was last seen for treatment purposes by the particular user or a time that the particular user last accessed patient data relevant to the clinical patient.

23. The user interface of claim 19, further showing a current time and date stamp display area.

24. The user interface of claim 23, further showing a time and date stamp selection region operative to receive a request to assign a new time and date stamp to the treatment data.

25. The user interface of claim 21, further showing a since last time display area, wherein the since last time display area contains the treatment data, the sections, and the attributes.

26. The user interface of claim 25, wherein the since last time display area is a since last time user interface tab.
